(19) United States
(12) Patent Application Publication
(10) Pub. No.: US 2021/0215653 A1
(43) Pub. Date: Jul. 15, 2021

(54) METHODS FOR ESTIMATING THE CONCENTRATIONS OF GAS-PHASE SPECIES WITHIN A PROCESS VESSEL FROM DOWNSTREAM SAMPLES

(71) Applicant: ExxonMobil Research and Engineering Company, Annandale, NJ (US)

(72) Inventors: Yira Y. Aponte Torrealba, Sarnia (CA); John Dusseault, Sarnia (CA)

(21) Appl. No.: 16/743,255

(22) Filed: Jan. 15, 2020

(51) Int. Cl. G01N 33/00

(52) U.S. Cl. CPC G01N 33/0013; G01N 33/0027; G01N 33/0062

(57) ABSTRACT

A method for estimating a gas concentration of a compound of interest within a process vessel for permitting safe entry into the process vessel comprises: exposing the process vessel to a condensing fluid to dissolve gases therein; collecting liquid comprising the condensing fluid from a location downstream of the process vessel; sealing the liquid in a sample container to yield a condensate sample; measuring and/or calculating properties of a liquid portion and a gas portion of the condensate sample; calculating a process vessel condensate density; determining a thermodynamic equilibrium correction factor; calculating the gas concentration of the compound of interest in the process vessel based on the properties of the condensate sample, a thermodynamic equilibrium correction factor, the process vessel condensate density, and optionally, a safety factor; and determining when the calculated gas concentration of the compound of interest in the process vessel permits safe entry into the process vessel.

### FIELD

The present disclosure relates to estimating the concentrations of gas-phase species within a process vessel from downstream samples.

### BACKGROUND

Petrochemical refining operations are capital intensive and require extensive preventative maintenance measures. For example, complex industrial systems such as refineries periodically shut down systems to perform preventative maintenance. The process downtime from preventative maintenance procedures is very costly in the form of lost revenue.

Generally, in a shutdown procedure, process equipment contains residual chemicals in the gas phase that could be harmful to workers, the system or a portion thereof is treated with water (or another comparable solvent) in the form of steam that facilitates liberation and transport of the gases. The steam is then condensed, thereby concentrating the chemical in the condensed phase.

During a shutdown, large process vessels may be chemically cleaned and undergo maintenance. In some instances, these actions require workers to physically enter process vessels. Before workers may enter a process vessel safely, the concentration of harmful chemicals in the gas phase (e.g., H₂S, benzene, and methane) within the process vessel must be confirmed to be at or below safe flammability and/or exposure limits. Measuring the gas concentrations within the process vessel directly are difficult, costly, and potentially dangerous. Typically, samples are taken from remote locations downstream of the process vessel. The concentration of harmful chemicals in the downstream samples is then assumed to be similar to the concentrations of harmful chemicals remaining within the process vessels.

### SUMMARY

The present disclosure relates to estimating the concentrations of gas-phase species within a process vessel from downstream samples.

A nonlimiting example method for estimating a gas concentration of a compound of interest within a process vessel for permitting safe entry into the process vessel of the present disclosure comprises: exposing the process vessel to a condensing fluid to dissolve gases therein; determining a chemical reaction within the process vessel has achieved a steady state condition; measuring a process vessel temperature and a process vessel pressure; collecting liquid comprising the condensing fluid from a location downstream of the process vessel; sealing the liquid in a sample container to yield a condensate sample comprising a liquid portion and a gas portion; measuring properties of the liquid portion of the condensate sample including: (a) a volume, (b) a mass, (c) a condensate temperature, and (d) a condensate pH; measuring a gas concentration of the compound of interest from the gas portion of the condensate sample; calculating properties of the liquid portion of the condensate sample including: (a) a density and (b) an amount of the compound of interest in solution; calculating a process vessel condensate density based on the properties of the condensate sample, the process vessel temperature, and the process vessel pressure; determining a thermodynamic equilibrium correction factor based on the condensate temperature, the condensate pH, and the amount of the compound of interest dissolved in the liquid portion of the condensate sample; calculating the gas concentration of the compound of interest in the process vessel based on the properties of the condensate sample, a thermodynamic equilibrium correction factor, the process vessel condensate density, and optionally, a safety factor; and determining when the calculated gas concentration of the compound of interest in the process vessel permits safe entry into the process vessel.

A nonlimiting example system of the present disclosure comprises: a processor; a memory coupled to the processor; and instructions provided to the memory, wherein the instructions are executable by the processor to: (A) receive measurements and/or derive values for properties of the condensate sample that include (I) properties of a liquid portion of a condensate sample that comprise (a) a volume, (b) a mass, (c) a condensate temperature, (d) a condensate pH, (e) a density, and (f) an amount of the compound of interest in solution, and (II) a gas concentration of the compound of interest from a gas portion of the condensate sample; calculating a process vessel condensate density based on the properties of the condensate sample, the process vessel temperature, and the process vessel pressure; (B) determine a thermodynamic equilibrium correction factor based on the condensate temperature, the condensate pH, and the amount of the compound of interest dissolved in the liquid portion of the condensate sample; (C) calculate the gas concentration of the compound of interest in the process vessel based on the properties of the condensate sample, the thermodynamic equilibrium correction factor, the process vessel condensate density, and optionally, a safety factor; and (D) determine when the calculated gas concentration of the compound of interest in the process vessel permits safe entry into the process vessel.

### BRIEF DESCRIPTION OF THE DRAWINGS

The following figures are included to illustrate certain aspects of the disclosure, and should not be viewed as exclusive configurations. The subject matter disclosed is capable of considerable modifications, alterations, combinations, and equivalents in form and function, as will occur to those skilled in the art and having the benefit of this disclosure.

FIG. 1 illustrates a flowchart of an example method.

FIG. 2 depicts a chart illustrating the conversion of a H₂S concentration from a clear condensate sample having a pH of 8 to conditions in a process vessel.

FIG. 3 depicts a chart illustrating the conversion of a H₂S concentration from a clear condensate sample having a pH of 6 to conditions in a process vessel.

FIG. 4 depicts a chart illustrating the conversion of a H₂S concentration from a dark condensate sample having a pH of 8 to conditions in a process vessel.

FIG. 5 depicts a chart illustrating the conversion of a H₂S concentration from a dark condensate sample having a pH of 6 to conditions in a process vessel.

FIG. 6 depicts a chart illustrating the conversion of a lower explosive limit (LEL) concentration from a clear condensate sample of pure methane and diethylbenzene to conditions in a process vessel.

### DETAILED DESCRIPTION

The present disclosure relates to estimating the concentrations of gas-phase species within a process vessel from downstream samples.

As described above, current practice assumes the gas composition at remote locations downstream of the process vessel (referred to herein as a sampling vessel) are the same as the gas composition in the process vessel. However, in reality, the gas compositions are not the same because the volume, temperature, pressure, and other conditions of the two vessels are different. In fact, the concentration of gas species like H₂S, benzene, and methane are often higher in the sampling vessel than in the process vessel. Therefore, when this occurs, the current practice would have a longer wait time before cleaning and/or maintenance can be performed on the process vessel.

The methods and systems described herein mathematically relate the composition and properties of a sample taken downstream of a process vessel to the gas composition at the process vessel. Such a mathematical relationship is based on Henry's law and thermodynamics. Advantageously, such methods and systems allows for expedited, safe entry into process vessels, which reduces the shutdown time and related expense.

Generally, the methods of the present disclosure are performed after a process vessel has been exposed to a condensing fluid to dissolve gases therein from the chemical process that, when online, occurs in the process vessel. As used herein, the term “condensing fluid” refers to molecules that are liquid from 5° C. to 40° C. Water is a preferred condensing fluid because of availability and ease of use, but other condensing fluids include, but are not limited to, methanol and ethanol.

The condensing fluid is conveyed to a sampling vessel downstream of the process vessel. The sampling vessel is an object (e.g., a drum, a pipe, a line, or the like) that contains the condensing fluid and is downstream of the process vessel. Herein unless otherwise specified, the term “condensing fluid” does not imply the phase of the fluid, which may be in gas phase, liquid phase, or both.

Then, the methods of the present disclosure comprise collecting liquid from the sampling vessel downstream of the process vessel, maintaining the liquid in a closed container to produce a condensate sample, measuring the composition and properties of the condensate sample, and estimating the concentration of one or more gaseous species in the process vessel based on the composition and properties of the sample. In the closed container, the liquid collected downstream of the process vessel comes to equilibrium with the gas phase in the container. Therefore, the condensate sample includes a gas phase portion and a liquid phase portion (primarily comprising the condensing fluid) having gases dissolved therein. There is likely little to no solid phase components in the condensate sample.

The method should only be applied in cases where the following three conditions are met. First, the system must have reached a steady state. As applied in the present disclosure steady state comprises: (a) no consumption of chemicals within the process vessel for a period of three consecutive measurements and/or at least three hours; (b) a temperature variation of 5° C. for at least two hours or less and pressure variation of 5 psig or less for at least three hours; and (c) consistent sample appearance by visual inspection. For example, visual inspection may reveal that the condensate sample has no visible hydrocarbon dispersed therein when transparent or has hydrocarbon traces when dark, brown, and/or cloudy. Second, total volume (gas and liquid portions) of condensate samples are accurately known. Third, the condensate samples are from a representative sampling point with a measured pH less than 10 (preferably about 5 to about 10, more preferably about 6 to about 8) and a temperature between about 5° C. and about 40° C. (preferably about 20° C. and about 30° C.).

Regarding the present disclosure, the method is contemplated for estimating the gas concentrations for hydrogen sulfide and the lower explosive limits of short chain hydrocarbons (e.g., C₉ and below) as pertaining to petrochemical refining. The method disclosed herein may be extended to other applications where a free steaming process is involved and the corresponding sampling comprises: (1) collection (e.g., sampling) of condensates in liquid phase; (2) known gas concentration of the compound of interest in the liquid sample's gas phase; and (3) there are no chemical reactions occurring in the condensate sample.

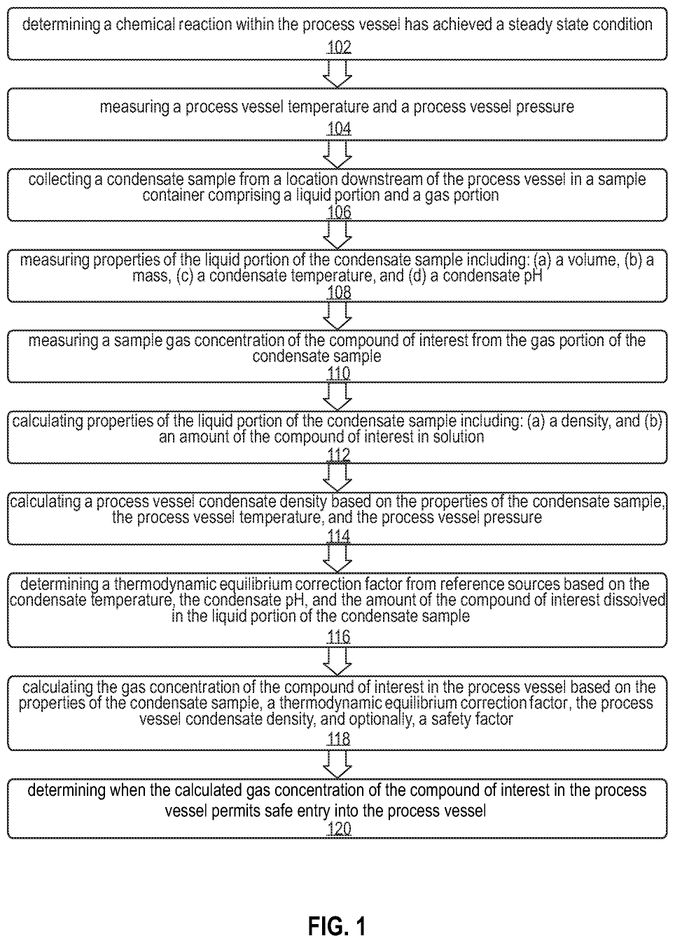

FIG. 1 illustrates a flow chart depicting a sample method 100 for determining when an estimated gas concentration of a compound of interest permits safe entry into a process vessel from a condensate sample collected downstream of the process vessel. The method 100 begins with determining that a chemical reaction within the process vessel has achieved a steady state condition in step 102. As noted earlier, steady state condition means no chemical consumption within the process vessel for three consecutive measurements and/or at least three hours, consistent process vessel temperature and pressure, and consistent condensate sample appearance. As will be discussed later, some assumptions are based on the sample appearance. In relation to the examples provided, assumptions about compositions are based on condensate samples that are clear or dark. This condition for determining steady state may require adjustment for applications outside of petrochemical refining. Next, a process vessel temperature and pressure is measured and recorded in step 104. A condensate sample is then collected from a location downstream of the process vessel in a sealable sample container of known total volume in step 106. The sample container must be sealable to allow for the measurement of a liquid portion and a gas portion of the various condensate properties. Measurements of the liquid portion of the condensate sample comprise: volume, mass, temperature, and pH in step 108. Next, the concentration of the compound of interest is measured in the gas portion of the condensate sample in step 110.

One skilled in the art will recognize suitable sensors, instruments, and/or methods for measuring a concentration of compounds of interest (e.g., single-gas or multi-gas sensors/instruments). Examples include, but are not limited to, gas chromatographs (e.g., with flame-ionization detectors, thermal conductivity detectors, and/or flame photometric detectors), electrochemical sensors, capillary-controlled sensors, infrared spectrometers, ultra-violet/visible spectrometers, and the like, and any combination thereof. The instruments are preferably field-usable (e.g., hand-held gas monitors) but laboratory-based instruments are also applicable.

It is contemplated that the sample container is sealed and of known volume, thus allowing the volume of the gas portion to be determined from the measurement of the liquid portion of the condensate sample. Furthermore, the temperature of the condensate sample should be fixed as the equilibrium gas concentration will change as the temperature changes. Monitoring the pH of the liquid portion of the condensate sample is necessary as the equilibrium conditions of the compound of interest may vary with pH values.

Next, properties of the liquid portion of the condensate sample are calculated including a density and an amount of the compound of interest in solution in step 112. Densities and other relevant properties may be calculated using process simulation tools/software with the appropriate thermodynamics package. Examples of such simulation tools/software include, but are not limited to, Pro/II (available from Aveva), Aspen Plus (available from Aspen Technology, Inc.), Aspen HYSYS (available from Aspen Technology, Inc.), and the like, and any hybrid or combination thereof.

In regards to the present disclosure the following assumptions were made in calculating the densities. The liquid portion of the condensate sample may be assumed to be 99.9% pure liquid water (or other condensing fluid if used) when the condensate sample is observed to be clear or substantially transparent. When the liquid portion of the condensate sample is observed to be dark and/or substantially not transparent it is assumed that a defined percentage of hydrocarbons are present. One of ordinary skill in the art would understand and possess knowledge of the chemical reactions taking place within the process vessel to determine what assumptions about the composition of the liquid portion of the condensate sample are necessary for the appropriate density calculation. It is contemplated that the method disclosed herein may be applied to many chemical reactions/relationships beyond the examples relating to petrochemical refining provided.

Next, a process vessel condensate density is calculated based on the properties of the condensate sample, the process vessel temperature, and the process vessel pressure in step 114. The process vessel is assumed to be at steady state as noted above and completely within the gas phase. Next, a thermodynamic equilibrium correction factor is determined (e.g., from reference sources) based on the properties of the condensate sample in step 116. The relevant properties comprise the temperature, pH, and amount of the compound of interest dissolved in solution of the liquid portion of the condensate sample. One skilled in the art will recognize the reference sources where components of the thermodynamic equilibrium correction factor can be obtained. Said sources may include graphs, tables, equations, or the like for the concentration of a species dissolved in the condensing liquid as a function of temperature, pH, pressure, or the like. In a more specific example, an article by Frank J. Millero in Marine Chemistry, 18 (1986) 121-147 includes a figure for the equilibrium concentration of sulfur species in aqueous phase as a function of pH for at 25° C. and 1 atm.

Next, a gas concentration of the compound of interest in the process vessel is calculated based on the properties of the condensate sample, the thermodynamic equilibrium correction factor, the process vessel condensate density, and optionally, a safety factor in step 118. Once again, the calculations are completed using process simulation tools/software with the appropriate thermodynamics package.

Next, safe entry into the process vessel is determined when the calculated gas concentration of the compound of interest in the process vessel is below the safe limit for the compound of interest in step 120.

The relationship between the gas concentration of the compound of interest in the process vessel and the condensate sample is depicted in Eq. 1 below.

$${[ gas ]}_{VESSEL} = {[ gas ]}_{sample}^{gp} * \frac{( {V}_{b} - {V}_{cond} )}{1 \cdot {10}^{6}} * ( \frac{1}{{R}_{{gas}_{pH}}} + {F}_{{gas}_{T}} + {E}_{gas} ) \frac{{m}_{cond}}{SF \cdot {ρ}_{vc}} \qquad Eq . 1$$

where:
[gas]_{VESSEL}=gas concentration within the process vessel, ppmᵥ;
[gas]^{gp}ₛₐₘₚₗₑ=gas concentration within the sample container, ppmᵥ;
V_{cond}=volume of liquid portion of condensate sample, mL;
V_{b}=total volume of the sample container, mL;
R_{gas}_{_{pH}}=correction factor for gas fraction in the condensate by pH effect, vol %;
F_{gas}_{_{T}}=correction factor for gas fraction dissolved in the condensate by temperature effect, vol %;
E_{gas}=correction factor for gas fraction of gas dissolved as an ion, vol %;
m_{cond}=mass of the condensate, g;
SF=safety factor, unitless; and
ρ_{vc}=condensate density calculated at the process vessel conditions, g/mL.

In Eq. 1, the thermodynamic equilibrium correction factor is the combination of the correction factors (R_{gaspH}, F_{gasT}, and E_{gas}) as shown in Eq. 1 above. For example, R_{gaspH} in a system with H₂S being the compound of interest and water being the condensing fluid is the amount of H₂S dissolved as HS⁻ in the water at pH of sample.

The safety factor as provided in Eq. 1 is greater than zero and less than or equal to one. The safety factor increases the concentration estimate [gas]_{VESSEL}, which essentially overestimates the concentration of said gas in the process vessel. Because operators look to reduce the concentration of the gas in the process value to a maximum level, the safety factor accounts for a desired level of risk management for the operator.

The methods and systems described herein may be applied to process vessels having a pressure of about 10 psi to about 150 psi, (or about 15 psi to about 35 psi, or about 10 psi to about 40 psi, or about 25 psi to 100 psi, or about 50 psi to about 150 psi).

The methods and systems described herein may also be applied to process vessels having a temperature of about 250° F. to about 350° F. (or about 250° F. to about 300° F., or about 300° F. to 350° F.).

The methods described herein may be applied to condensate samples containing hydrocarbons dissolved in solution of about 2% to about 10% (or about 3% to about 5%, or about 5% to about 7%).

The methods described herein may be applied to process vessels incorporated into many known processes and systems. Some example processes utilized in petrochemical refining systems include, but are not limited to, fluid catalytic cracking, fixed bed reactors, amine regeneration, heat transfer equipment, pumps, compressors, storage tanks, drums, piping, piping systems, and any distillation or separation towers. One having ordinary skill in the art would understand the chemical and thermodynamic principles required to apply the methods disclosed herein to systems and processes outside of the examples provided.

The methods described herein may be performed with the assistance of a computer or other processor-based device.

“Computer-readable medium” or “non-transitory, computer-readable medium,” as used herein, refers to any non-transitory storage and/or transmission medium that participates in providing instructions to a processor for execution. Such a medium may include, but is not limited to, non-volatile media and volatile media. Non-volatile media includes, for example, NVRAM, or magnetic or optical disks. Volatile media includes dynamic memory, such as main memory. Common forms of computer-readable media include, for example, a floppy disk, a flexible disk, a hard disk, an array of hard disks, a magnetic tape, or any other magnetic medium, magneto-optical medium, a CD-ROM, a holographic medium, any other optical medium, a RAM, a PROM, an EPROM, a FLASH-EPROM, a solid state medium like a memory card, any other memory chip or cartridge, or any other tangible medium from which a computer can read data or instructions. When the computer-readable media is configured as a database, it is to be understood that the database may be any type of database, such as relational, hierarchical, object-oriented, and/or the like. Accordingly, exemplary embodiments of the present systems and methods may be considered to include a tangible storage medium or tangible distribution medium and prior art-recognized equivalents and successor media, in which the software implementations embodying the present techniques are stored.

The methods described herein can, and in many embodiments must, be performed using computing devices or processor-based devices that include a processor; a memory coupled to the processor; and instructions provided to the memory, wherein the instructions are executable by the processor to perform the methods described herein (such computing or processor-based devices may be referred to generally by the shorthand “computer”). For example, a system may comprise: a processor; a memory coupled to the processor; and instructions provided to the memory, wherein the instructions are executable by the processor to (A) receive measurements and/or derive values for properties of the condensate sample that include (I) properties of a liquid portion of a condensate sample that comprise (a) a volume, (b) a mass, (c) a condensate temperature, (d) a condensate pH, (e) a density, and (f) an amount of the compound of interest in solution, and (II) a gas concentration of the compound of interest from a gas portion of the condensate sample; calculating a process vessel condensate density based on the properties of the condensate sample, the process vessel temperature, and the process vessel pressure; (B) determine a thermodynamic equilibrium correction factor based on the condensate temperature, the condensate pH, and the amount of the compound of interest dissolved in the liquid portion of the condensate sample; (C) calculate the gas concentration of the compound of interest in the process vessel based on the properties of the condensate sample, the thermodynamic equilibrium correction factor, the process vessel condensate density, and optionally, a safety factor; and (D) determine when the calculated gas concentration of the compound of interest in the process vessel permits safe entry into the process vessel.

Similarly, any calculation, determination, or analysis recited as part of methods described herein may be carried out in whole or in part using a computer.

Furthermore, the instructions of such computing devices or processor-based devices can be a portion of code on a non-transitory computer readable medium. Any suitable processor-based device may be utilized for implementing all or a portion of embodiments of the present techniques, including without limitation personal computers, network personal computers, laptop computers, computer workstations, mobile devices, multi-processor servers or workstations with (or without) shared memory, high performance computers, and the like. Moreover, embodiments may be implemented on application specific integrated circuits (ASICs) or very large scale integrated (VLSI) circuits.

### Example Embodiments

A nonlimiting example method for estimating a gas concentration of a compound of interest within a process vessel for permitting safe entry into the process vessel comprises: exposing the process vessel to a condensing fluid to dissolve gases therein; determining a chemical reaction within the process vessel has achieved a steady state condition; measuring a process vessel temperature and a process vessel pressure; collecting liquid comprising the condensing fluid from a location downstream of the process vessel; sealing the liquid in a sample container to yield a condensate sample comprising a liquid portion and a gas portion; measuring properties of the liquid portion of the condensate sample including: (a) a volume, (b) a mass, (c) a condensate temperature, and (d) a condensate pH; measuring a gas concentration of the compound of interest from the gas portion of the condensate sample; calculating properties of the liquid portion of the condensate sample including: (a) a density and (b) an amount of the compound of interest in solution; calculating a process vessel condensate density based on the properties of the condensate sample, the process vessel temperature, and the process vessel pressure; determining a thermodynamic equilibrium correction factor (e.g., from reference sources) based on the condensate temperature, the condensate pH, and the amount of the compound of interest dissolved in the liquid portion of the condensate sample; calculating the gas concentration of the compound of interest in the process vessel based on the properties of the condensate sample, a thermodynamic equilibrium correction factor, the process vessel condensate density, and optionally, a safety factor; and determining when the calculated gas concentration of the compound of interest in the process vessel permits safe entry into the process vessel. The nonlimiting example method may further include one or more of: Element 1: wherein the steady state condition comprises no consumption of chemicals within the process vessel during a period of three consecutive measurements and/or at least three hours; Element 2: wherein the measured pH of the liquid portion of the condensate sample is less than 10; Element 3: wherein the measured pH of the liquid portion of the condensate sample is about 6 to about 8; Element 4: wherein the measured temperature of the liquid portion of the condensate sample is between about 5° C. to about 40° C.; Element 5: wherein the measured temperature of the liquid portion of the condensate sample is between about 20° C. to about 30° C.; Element 6: wherein the condensate sample comprises about 2% and about 10% dissolved hydrocarbons; Element 7: wherein the condensate sample comprises about 3% and about 5% dissolved hydrocarbons; Element 8: wherein the process vessel temperature is between about 250° F. and about 350° F.; Element 9: wherein the process vessel temperature is between about 250° F. and about 300° F.; Element 10: wherein the process vessel pressure is between about 10 psi and about 150 psi; Element 11: wherein the process vessel pressure is between about 15 psi and about 35 psi; Element 12: wherein the condensing fluid is water; Element 13: wherein the compound of interest is hydrogen sulfide; Element 14: wherein the compound of interest is hydrocarbons and the calculated gas concentration relates to a lower explosive limit concentration; Element 15 the method claim further comprising: entering the process vessel when the calculated gas concentration of the compound of interest in the process vessel permits safe entry into the process vessel; and Element 16: the method further comprising: performing a cleaning operation and/or maintenance in the process vessel when the calculated gas concentration of the compound of interest in the process vessel permits safe entry into the process vessel. Examples of combinations include, but are not limited to, Elements 13 and 14 in combination; Element 13 and/or Element 14 in combination with Element 12; two or more of Elements 2-11 in combination; one or more of Elements 2-11 in combination with one or more of Elements 12-14; Element 1 in combination with one or more of Elements 2-14; Element 15 and 16 in combination; and Element 15 and/or Element 16 in combination with one or more of Elements 1-14.

A nonlimiting example system for estimating a gas concentration of a compound of interest within a process vessel for permitting safe entry into the process vessel comprises: a processor; a memory coupled to the processor; and instructions provided to the memory, wherein the instructions are executable by the processor to: (A) receive measurements and/or derive values for properties of the condensate sample that include (I) properties of a liquid portion of a condensate sample that comprise (a) a volume, (b) a mass, (c) a condensate temperature, (d) a condensate pH, (e) a density, and (f) an amount of the compound of interest in solution, and (II) a gas concentration of the compound of interest from a gas portion of the condensate sample; calculating a process vessel condensate density based on the properties of the condensate sample, the process vessel temperature, and the process vessel pressure; (B) determine a thermodynamic equilibrium correction factor based on the condensate temperature, the condensate pH, and the amount of the compound of interest dissolved in the liquid portion of the condensate sample; (C) calculate the gas concentration of the compound of interest in the process vessel based on the properties of the condensate sample, the thermodynamic equilibrium correction factor, the process vessel condensate density, and optionally, a safety factor; and (D) determine when the calculated gas concentration of the compound of interest in the process vessel permits safe entry into the process vessel. The condensate sample comprises a condensing fluid (e.g., water). The compound of interest, for example, may be hydrogen sulfide and/or hydrocarbons.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the incarnations of the present invention. At the very least, and not as an attempt to limit the application of the doctrine of equivalents to the scope of the claim, each numerical parameter should at least be construed in light of the number of reported significant digits and by applying ordinary rounding techniques.

One or more illustrative incarnations incorporating the invention elements disclosed herein are presented herein. Not all features of a physical implementation are described or shown in this application for the sake of clarity. It is understood that in the development of a physical embodiment incorporating the embodiments of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While compositions and methods are described herein in terms of “comprising” various components or steps, the compositions and methods can also “consist essentially of” or “consist of” the various components and steps.

To facilitate a better understanding of the embodiments of the present invention, the following examples of preferred or representative embodiments are given. In no way should the following examples be read to limit, or to define, the scope of the invention.

### Examples

An example embodiment of the present disclosure is estimating a concentration of hydrogen sulfide (H₂S) gas within a process vessel after a chemical cleaning operation has been performed to determine when maintenance personnel may safely enter. Method 100 was applied utilizing the relationship expressed in Eq.1. The following four conditions were met for the data gathering process:

1. The measured H₂S concentration was stable for three consecutive readings over three hours during a vapor phase chemical cleaning.

2. The chemical consumption rate was stable (no consumption of chemicals) in the more than 3 consecutives measurements (or >3 h).

3. Samples were steam condensates from a representative downstream sampling point, with the following conditions:
i) Sample bottle volume was 250 mL;
ii) Sample aliquot was 125 mL; and
iii) Sample pH ranged between pH of 6-8.

4. The operating chemical process conditions of the vessel were within the ranges of Table 1.

TABLE 1

| Process Equip. | | |
|---|---|---|
| Conditions | Temperature (° F.) | Pressure (psi) |
| Minimum | 250 | 15 |
| Maximum | 300 | 50 |
| Target | 275 | 30 |

The relationship disclosed in Eq. 1 (safety factor equal to 0.75) was developed based on the data from operations within the limits disclosed in Table 1. Therefore, application of the method 100 and Eq. 1 should only be used when all the above conditions are met.

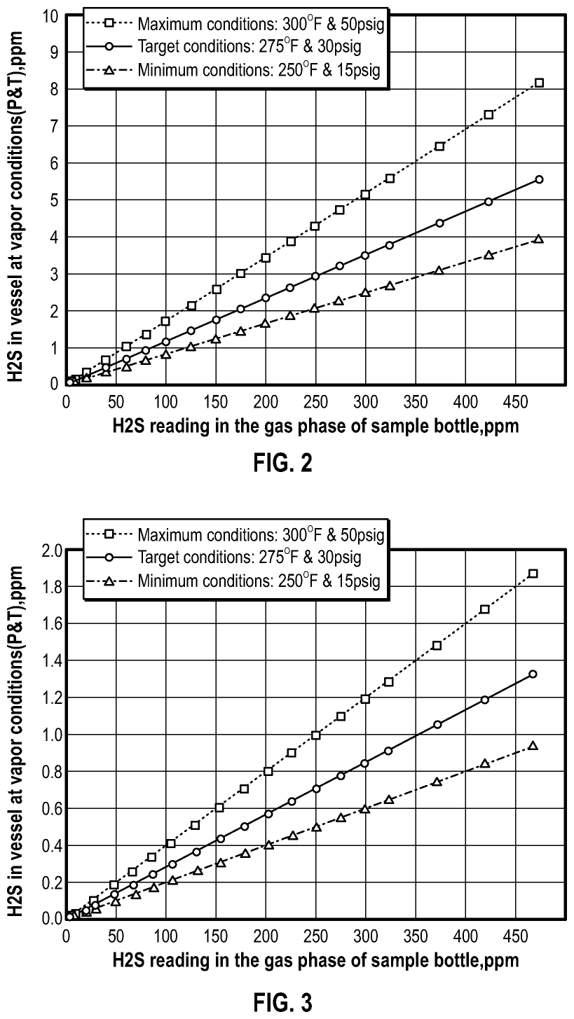

FIGS. 2 and 3 depict the correlations for H₂S gas concentration measurements from downstream condensate samples and estimated gas H₂S concentration in process vessels at chemical cleaning conditions. The condensate samples were clear, indicating no trace of hydrocarbons. The condensate samples collected for FIG. 2 had pH values of about 8. The condensate samples collected for FIG. 3 had pH values of about 6. The effect of condensate sample pH is illustrated by the differences between FIGS. 2 and 3.

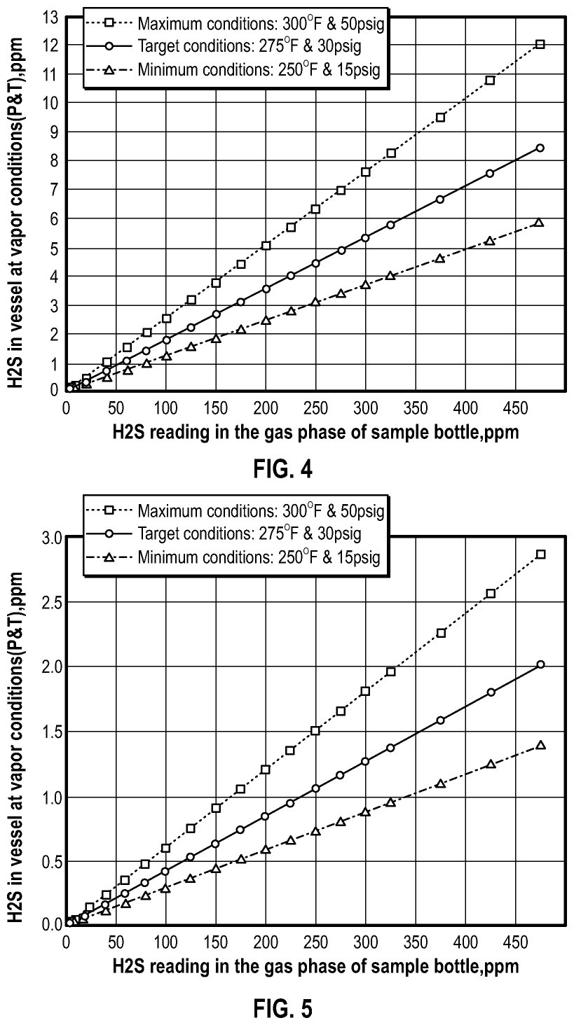

FIGS. 4 and 5 depict the correlations for H₂S gas concentration measurements from downstream condensate samples and estimated gas H₂S concentration in process vessels at chemical cleaning conditions for dark condensate samples. The dark appearance of the condensate samples is due to the presence of hydrocarbons dissolved in solution. For the application of the method 100 and Eq. 1, the condensate samples were assumed to contain 5% hydrocarbons dissolved in solution. Higher density of the dark samples results in higher H₂S concentrations in the process vessel when compared to the clear samples.

Table 2 below shows the results of experimental testing of the method 100 (safety factor equal to 0.75) applied to estimating H₂S concentrations in several process vessels undergoing chemical cleaning processes.

TABLE 2

| | | Sample | Estimated | Actual |
|---|---|---|---|---|
| | | H₂S | H₂S | H₂S vessel |
| | Sampling | readings, | concentration, | concentration |
| Equipment | points | ppm | ppm | measured, ppm |
| FCC Main | 2 | 11-12 | 0.25 | <5 |
| Fractionator | | | | |
| Sponge | 6 | 85-200 | 0.75-1.85 | <5 |
| Absorbers | | | | |
| and Drums | | | | |

The impact of the results shown in Table 2 was an estimated reduction in downtime of about 12 hours. The successful estimation of H₂S concentrations in the process vessels below safe limits permitted earlier entry into the process vessels and the value of the downtime saved is approximately CAD $1.3 million.

Similarly, Table 3 below shows the results of experimental testing of the method 100 (safety factor equal to 0.75) applied to estimating H₂S concentrations in another process vessel undergoing a chemical cleaning process.

TABLE 3

| | | Sample | Estimated | Actual |
|---|---|---|---|---|
| | | H₂S | H₂S | H₂S vessel |
| | Sampling | readings, | concentration, | concentration |
| Equipment | points | ppm | ppm | measured, ppm |
| MonoEthanolAmine | 1 | 40 | 2 | <5 |
| Regen Tower/drums | | | | |

The impact of the results shown in Table 3 was an estimated reduction in downtime of about 4 hours.

Another example embodiment of the present disclosure is estimating a lower explosive limit (LEL) of hydrocarbon gas within a process vessel after a chemical cleaning operation has to been performed to determine when maintenance personnel may safely enter. The same four operating conditions as described above in regards to H₂S apply were met in collecting samples for estimating LEL in process vessels. It should be noted, that method 100 requires an extra intermediate step of converting % LEL readings from the condensate sample into a concentration of a pure light hydrocarbon. Gas concentrations were taken from known standard LEL measurements.

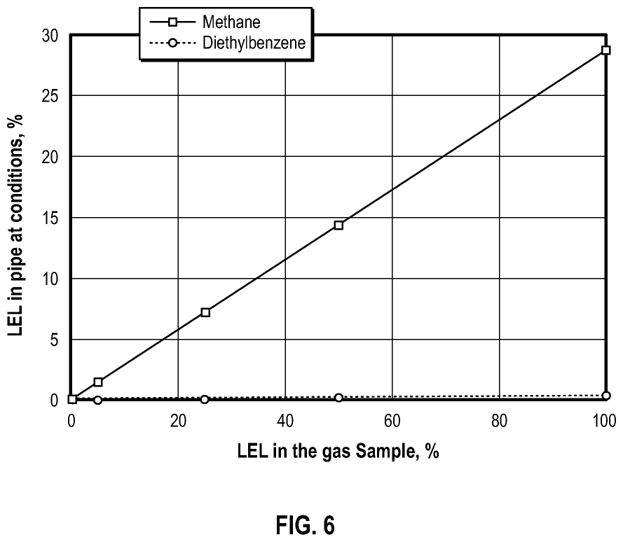

FIG. 6 depicts the correlation for LEL measurements from downstream condensate samples and estimated LEL in process vessels at chemical cleaning conditions. The samples collected for LEL analysis were clear condensate samples having no observed hydrocarbons.

Example claims describing the methods and systems disclosed herein include:

1. A method of estimating a gas concentration of a compound of interest within a process vessel for permitting safe entry into the process vessel, the method comprising:

exposing the process vessel to a condensing fluid to dissolve gases therein;

determining a chemical reaction within the process vessel has achieved a steady state condition;

measuring a process vessel temperature and a process vessel pressure;

collecting liquid comprising the condensing fluid from a location downstream of the process vessel;

sealing the liquid in a sample container to yield a condensate sample comprising a liquid portion and a gas portion;

measuring properties of the liquid portion of the condensate sample including: (a) a volume, (b) a mass, (c) a condensate temperature, and (d) a condensate pH;

measuring a gas concentration of the compound of interest from the gas portion of the condensate sample;

calculating properties of the liquid portion of the condensate sample including: (a) a density and (b) an amount of the compound of interest in solution;

calculating a process vessel condensate density based on the properties of the condensate sample, the process vessel temperature, and the process vessel pressure;

determining a thermodynamic equilibrium correction factor based on the condensate temperature, the condensate pH, and the amount of the compound of interest dissolved in the liquid portion of the condensate sample;

calculating the gas concentration of the compound of interest in the process vessel based on the properties of the condensate sample, a thermodynamic equilibrium correction factor, the process vessel condensate density, and optionally, a safety factor; and

determining when the calculated gas concentration of the compound of interest in the process vessel permits safe entry into the process vessel.

2. The method of claim 1, wherein the steady state condition comprises no consumption of chemicals within the process vessel during a period of three consecutive measurements and/or at least three hours.

3. The method of any preceding claim, wherein the measured pH of the liquid portion of the condensate sample is less than 10.

4. The method of any preceding claim, wherein the measured pH of the liquid portion of the condensate sample is about 6 to about 8.

5. The method of any preceding claim, wherein the condensing fluid is water.

6. The method of any preceding claim, wherein the measured temperature of the liquid portion of the condensate sample is between about 5° C. to about 40° C.

7. The method of any preceding claim, wherein the measured temperature of the liquid portion of the condensate sample is between about 20° C. to about 30° C.

8. The method of any preceding claim, wherein the condensate sample comprises about 2% and about 10% dissolved hydrocarbons.

9. The method of any preceding claim, wherein the condensate sample comprises about 3% and about 5% dissolved hydrocarbons.

10. The method of any preceding claim, wherein the process vessel temperature is between about 250° F. and about 350° F.

11. The method of any preceding claim, wherein the process vessel temperature is between about 250° F. and about 300° F.

12. The method of any preceding claim, wherein the process vessel pressure is between about 10 psi and about 150 psi.

13. The method of any preceding claim, wherein the process vessel pressure is between about 15 psi and about 35 psi.

14. The method of any preceding claim, wherein the compound of interest is hydrogen to sulfide.

15. The method of any preceding claim, wherein the compound of interest is hydrocarbons and the calculated gas concentration relates to a lower explosive limit concentration.

16. The method of any preceding claim further comprising:

entering the process vessel when the calculated gas concentration of the compound of interest in the process vessel permits safe entry into the process vessel.

17. The method of any preceding claim further comprising:

performing a cleaning operation and/or maintenance in the process vessel when the calculated gas concentration of the compound of interest in the process vessel permits safe entry into the process vessel.

18. A system comprising:

a processor; a memory coupled to the processor; and instructions provided to the memory, wherein the instructions are executable by the processor to:

receive measurements and/or derive values for properties of the condensate sample that include (I) properties of a liquid portion of a condensate sample that comprise (a) a volume, (b) a mass, (c) a condensate temperature, (d) a condensate pH, (e) a density, and (f) an amount of the compound of interest in solution, and (II) a gas concentration of the compound of interest from a gas portion of the condensate sample; calculating a process vessel condensate density based on the properties of the condensate sample, the process vessel temperature, and the process vessel pressure;

determine a thermodynamic equilibrium correction factor based on the condensate temperature, the condensate pH, and the amount of the compound of interest dissolved in the liquid portion of the condensate sample;

calculate the gas concentration of the compound of interest in the process vessel based on the properties of the condensate sample, the thermodynamic equilibrium correction factor, the process vessel condensate density, and optionally, a safety factor; and

determine when the calculated gas concentration of the compound of interest in the process vessel permits safe entry into the process vessel.

Therefore, the present invention is well adapted to attain the ends and advantages mentioned as well as those that are inherent therein. The particular examples disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative examples disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

1. A method of estimating a gas concentration of a compound of interest within a process vessel for permitting safe entry into the process vessel, the method comprising:
exposing the process vessel to a condensing fluid to dissolve gases therein;
determining a chemical reaction within the process vessel has achieved a steady state condition;
measuring a process vessel temperature and a process vessel pressure;
collecting liquid comprising the condensing fluid from a location downstream of the process vessel;
sealing the liquid in a sample container to yield a condensate sample comprising a liquid portion and a gas portion;
measuring properties of the liquid portion of the condensate sample including: (a) a volume, (b) a mass, (c) a condensate temperature, and (d) a condensate pH;
measuring a gas concentration of the compound of interest from the gas portion of the condensate sample;
calculating properties of the liquid portion of the condensate sample including: (a) a density and (b) an amount of the compound of interest in solution;
calculating a process vessel condensate density based on the properties of the condensate sample, the process vessel temperature, and the process vessel pressure;
determining a thermodynamic equilibrium correction factor based on the condensate temperature, the condensate pH, and the amount of the compound of interest dissolved in the liquid portion of the condensate sample;
calculating the gas concentration of the compound of interest in the process vessel based on the properties of the condensate sample, a thermodynamic equilibrium correction factor, the process vessel condensate density, and optionally, a safety factor; and
determining when the calculated gas concentration of the compound of interest in the process vessel permits safe entry into the process vessel.

2. A system comprising:
a processor; a memory coupled to the processor; and instructions provided to the memory, wherein the instructions are executable by the processor to:
receive measurements and/or derive values for properties of the condensate sample that include (I) properties of a liquid portion of a condensate sample that comprise (a) a volume, (b) a mass, (c) a condensate temperature, (d) a condensate pH, (e) a density, and (f) an amount of the compound of interest in solution, and (II) a gas concentration of the compound of interest from a gas portion of the condensate sample; calculating a process vessel condensate density based on the properties of the condensate sample, the process vessel temperature, and the process vessel pressure;
determine a thermodynamic equilibrium correction factor based on the condensate temperature, the condensate pH, and the amount of the compound of interest dissolved in the liquid portion of the condensate sample;
calculate the gas concentration of the compound of interest in the process vessel based on the properties of the condensate sample, the thermodynamic equilibrium correction factor, the process vessel condensate density, and optionally, a safety factor; and
determine when the calculated gas concentration of the compound of interest in the process vessel permits safe entry into the process vessel.
